(19) United States
(12) Patent Application Publication
(10) Pub. No.: US 2025/0359912 A1
(43) Pub. Date: Nov. 27, 2025

(54) SURGICAL ASSISTANCE DEVICE

(71) Applicant: W. Joseph GORUM, Wilmington, DE (US)

(72) Inventor: W. Joseph GORUM, Wilmington, DE (US)

(21) Appl. No.: 19/204,743

(22) Filed: May 12, 2025

Related U.S. Application Data
(60) Provisional application No. 63/649,980, filed on May 21, 2024.

(51) Int. Cl. A61B 17/90; A61B 17/00; A61B 17/88; A61M 25/09

(52) U.S. Cl. CPC A61B 17/90; A61B 17/8897; A61B 2017/00438; A61M 25/09041

(57) ABSTRACT

A surgical assistance device has a ring band that is worn on a surgeon's finger from which opposed clasp jaws extend. The opposed clasp jaws define a channel configured to receive a guidewire to be inserted into a patient's bone. The surgeon locates a desired entry point for the guidewire with his or her finger on which the ring band is worn, and thereby positions the channel of the device to receive the guidewire so that such guidewire when inserted through the channel is directed in the desired entry point and into the patient. The surgeon maintains his or her finger at the desired location during guidewire insertion, and then detaches the device from the installed guidewire by separating the opposed clasp jaws.

### CROSS-REFERENCE TO RELATED APPLICATION

This application claims priority under 35 USC § 119 (e) to U.S. Provisional Application Ser. No. 63/649,980, entitled “Surgical Assistance Device”, filed May 21, 2024, the disclosure of which is incorporated by reference herein.

### TECHNICAL FIELD

The present invention is generally directed to medical devices, and more specifically to an orthopedic surgical assistance device worn on a finger, for example, a surgeon's or other practitioner's forefinger and configured to orient a guidewire for insertion into a patient.

### BACKGROUND

Mini-incisions are a standard of care, even in patients with elevated BMI (Body Mass Index), because research has shown that there is significantly less tissue trauma from surgery with smaller incisions. Benefits to the patient include less post-surgery pain, a shorter recovery period, and less scarring. In many situations, it can be technically challenging to achieve such benefits, especially in patients with robust hips, based on how difficult it is to achieve proper positioning to insert and drill surgical implants, for example, such as guidewires, including long drillable guidewires.

When a surgeon or other practitioner fixes, for example, a femur fracture, after making an appropriate incision, he or she palpates the bone by hand to locate an optimum location for inserting a guidewire into the bone. Once so located, the guidewire is then drilled into the bone. It is difficult to insert the guidewire at the desired location and at the desired orientation while keeping one hand on the patient's femur bone and using the other hand to operate the drill with the guidewire, especially when the amount of adipose tissue and muscle attached and covering the tip of the femur bone significantly affects the drilling angle of the guidewire into the bone. The guidewire can slip out of position and, for example, off the femur bone. Imaging equipment is needed to facilitate drilling, adding complexity. The surgeon's glove can be pierced or torn by the guidewire and/or due to the slippage. Currently there is a void of medical devices that function as intraoperative surgical assistants specifically designed for fracture care in injured patients.

One proposal to address this problem is disclosed in Chinese utility model CN211934259U. An auxiliary Kirschner wire imbedding device has a thumb ring and a finger ring that each have squeeze plates arranged thereon. Grooves in each squeeze plate align when the opposing faces of the plates are brought together by squeezing the thumb ring and finger ring toward one another, thereby to form a guide that holds the Kirschner wire in the aligned grooves between the squeeze plates. The proposed device is cumbersome due to the shape of the plates and the need for the clinician to have rings on both a finger and a thumb. The guidewire position is not maintained if gripping pressure is released between the finger and thumb. This device requires two fingers to hold the two pressing plates, one on the thumb and the other on the index finger and to squeeze pressing plates with a groove to help with Kirshner wire insertion. The proposed device is cumbersome due to the shape of the plates and requires the clinician to have two rings on one on a finger and the other on a thumb. This device is not intended to be used during surgery and does not fit inside a patient's mini-incision.

U.S. Pat. No. 8,142,416 B2 discloses a guide wire introducer with integrated wire steering function for treating bone. The guide wire employed with the introducer includes a bore material that is a pseudo-elastic metal alloy, such as Nitinol. This introducer has a chamfered tip with the complement of the outer construction including a uniform narrow outer diameter enabling insertion into soft tissue and bone with a guide wire therein. The chamfered tip of the introducer incorporates as integrated slit that enables the steering function of the introducer. Additionally, the outer surface of the introducer incorporates a radiolucent notch for radial position reference.

WO 2014/162459 A1 describes a medical aid having a mounting part which is mounted on a finger that wears a medical glove on top of the medical glove, and a picking part which is attached to the mounting part and extends toward a distal end along the finger from the mounting part. The length of the picking part in the lateral direction is greater than the thickness of the picking part. The thickness of the picking part tapers from the base end to the distal end. The medical aid is used when holding a wire-like medical tool. The picking part functions as an extension to the clinician's forefinger to facilitate picking up or scraping a wire.

Other finger-mounted surgical aids are addressed to different concerns that do not involve guidewires. For example, US 2004/0193211 A1 shows a minimally invasive surgical instrument that may be used in hand-assisted laparoscopic surgeries. The device is multifunctional surgical instrument that may be mounted directly on a surgeon's fingertip and inserted through an incision to allow the surgeon to manipulate tissue during a surgical procedure.

WO 2004/073495 A2 shows a surgical instrument that may be used in hand-assisted laparoscopic surgeries. The device is a multifunctional surgical instrument that may be mounted directly on a surgeon's fingertip and inserted through an incision to allow the surgeon to manipulate tissue during a surgical procedure.

U.S. Pat. No. 5,925,064 shows a series of surgical instruments mounted directly on a surgeon's fingertips in a way that the surgeon can insert his or her hand into a patient through a minimal incision to perform surgical procedures, and also to use his or her fingers to manipulate tissues. The invention enables the surgeon to perform the procedures with all the benefits of minimally invasive surgery, but with much greater tactile sense, control, and ease of manipulation, than enabled by known minimally invasive surgical instruments.

Improvements to aid surgeons or other clinicians to effectively locate and insert guidewires into desired locations of the bones of surgical patients continue to be sought. None of the aforementioned devices are approved or indicated for use in orthopedic surgery to assist with the intraoperative treatment of bone injuries.

### SUMMARY

A surgical assistance device has a substantially ring-shaped band (continuous or discontinuous) from which outwardly extends a first clasp jaw and a second clasp jaw. The first clasp jaw has a prong end and the second clasp jaw in one embodiment has two prong ends: a first prong end and a second prong end. In such embodiment, the first prong end and the second prong end are spaced apart. At least a portion of the first clasp jaw is configured to be received in the space between the first prong end and the second prong end of the second clasp jaw. When the first clasp jaw and the second clasp jaw are so engaged together a channel configured to receive a surgical guidewire is formed. One or more of the prong end of the first clasp jaw and the first prong end and the second prong end of the second clasp jaw are movable in relation to one another. Such movement permits the opposing prong ends to be separated or spaced apart a sufficient distance to create an opening in the channel so that the guidewire can be extracted from the channel.

In an advantageous embodiment of the surgical assistance device, the band defines an elongated slot, and a proximal portion of the first clasp jaw is swivelably or pivotably held in the elongated slot of the band. In this embodiment, the first prong end and the second prong end of the second clasp jaw have proximal ends extending from the band at opposite sides of the elongated slot.

In an especially advantageous embodiment of the surgical assistance device the first clasp jaw and/or the second clasp jaw swivel or pivot when opposing external circumferential surfaces of the band are squeezed. In this manner, the surgeon or other clinician may retain the band on their forefinger and squeeze opposing sides of the band to separate the prong ends of the first and second clasp jaws and thereby open the channel so that a guidewire may be extracted from the channel. In other words, the surgical assistance device may be extracted from the patient while the guidewire remains in the patient even without removing the band of the surgical assistance device from the surgeon's or clinician's finger.

The prong end of the first clasp jaw may opposingly face the first prong end and the second prong end of the second clasp jaw when said prong end and said first prong end and second prong end are separated from one another.

In one embodiment of the surgical assistance device the proximal ends of the first clasp jaw and the second clasp jaw extend substantially radially outwardly from the band. In such embodiment, the band defines a center axis, and the channel defines a channel center axis, with the channel center axis substantially parallel to the center axis of the band. With this embodiment, the threaded guidewire may be advanced in a direction that is substantially parallel to the surgeon's or other clinician's finger that is wearing the band.

The band, the first clasp jaw and the second clasp jaw may be integrally formed of a same material. In one embodiment, the surgical assistance device is formed of a metal or medical-grade plastic that can withstand medical sterilization at a temperature of about 270° F. or above. Representative metals are stainless steel, aluminum and alloys thereof.

In still another advantageous embodiment of the invention, a surgical assistance device has a substantially ring-shaped band. A first clasp jaw extends outwardly from the band and has a prong end. A second clasp jaw extends outwardly from the band and has a first prong end. The prong end of the first clasp jaw opposingly faces the first prong end of the second clasp jaw, with the first clasp jaw and the second clasp jaw when engaged together or in contact with one another forming a channel configured to receive a guidewire. One or more of the prong end of the first clasp jaw and the first prong end of the second clasp jaw are movable in relation to one another. Optionally, the band defines an elongated slot, and a proximal portion of the first clasp jaw is swivelably or pivotably held in the elongated slot of the band. In this embodiment, the band defines a center axis, and the channel defines a channel center axis, and the channel center axis is substantially parallel to the center axis of the band.

Just as with earlier embodiments, the band, the first clasp jaw and the second clasp jaw may be integrally formed of a same material. The surgical assistance can be formed of a metal that can withstand medical sterilization at a temperature of about 270° F. or above, such as but not limited to, stainless steel or aluminum or alloys thereof.

Another aspect of the invention is a method of placing and advancing a surgical guidewire. The steps of a method may include, for example, wearing a surgical assistance device according to the invention on a forefinger or gloved forefinger, with the first clasp jaw and the second clasp jaw engaged together. The method may further include the step of inserting the forefinger or gloved forefinger through an incision in a patient to determine by feel a desired location for guidewire insertion into a bone of the patient. Additionally, the method may include the step of threading the guidewire through the channel of the surgical assistance device and advancing the guidewire into the bone of the patient. Additionally, the method may include the steps of squeezing opposing sides of the band to swivel or pivot one or both of the first clasp jaw and the second clasp jaw to separate the prong of the first clasp jaw from the first prong and the second prong of the second clasp jaw to open the channel and detaching the channel from the guidewire.

In a variant of the inventive method, the prong end of the first clasp jaw opposingly faces the first prong end and the second prong end of the second clasp jaw when said prong end and said first prong end and second prong end are separated from one another.

It is envisioned that the proximal ends of the first clasp jaw and the second clasp jaw may extend substantially radially outwardly from the band. In this configuration, the band defines a center axis, wherein the channel defines a channel center axis, and the channel center axis is substantially parallel to the center axis of the band. The guidewire then may be threaded in the direction of the channel center axis, which is a direction substantially parallel to the forefinger of the surgeon or clinician wearing band of the surgical assistance device.

### BRIEF DESCRIPTION OF THE DRAWINGS

The foregoing summary, as well as the following detailed description of the disclosure, will be better understood when read in conjunction with the appended drawings. For the purpose of illustrating the disclosure, there is shown in the drawings an embodiment of a surgical assistance device and an embodiment of a method of placing and advancing a surgical guidewire that are presently preferred. It should be understood, however, that the disclosure is not limited to the precise arrangements and instrumentalities shown.

In the drawings:

FIG. 1 is a front elevational view of a surgical assistance device according to the invention;

FIG. 2 is a left side elevational view of the surgical assistance device of FIG. 1;

FIG. 3 is a schematic view of a first step of a surgical method wherein an incision is made in a patient to provide access to the patient's bone for treatment;

FIG. 4 is a schematic view of a second step of the surgical method in which a surgeon or other clinician has inserted a gloved forefinger on which the surgical assistance device is worn into the patient to locate desired entry point for a guidewire;

FIG. 5 is a schematic view of a third step of the surgical method in which a guidewire is inserted into the channel of the surgical assistance device;

FIG. 6 is a schematic view of a fourth step of the surgical method in which the guidewire is advanced through the surgical assistance device and into the patient's bone;

FIG. 7 is a schematic view of a fifth step of the surgical method in which prongs of the clasp jaws forming the channel are separated to open the channel;

FIG. 8 is a schematic view of a sixth step of the surgical method in which the surgical assistance device is separated from the guidewire and extracted from the patient; and

FIG. 9 is a flowchart of a method of performing a surgical procedure with an exemplary embodiment of a surgical assistance device, as described herein.

### DETAILED DESCRIPTION

It should be appreciated that the present invention provides practitioners with a sense of feel and function by providing a targeted drill guide for the optimum starting point to expedite and facilitate orienting a guidewire for insertion into a patient, and more specifically a tip (such as the trochanteric entry point) of a patient's fractured bone. Additional objectives, advantages, features and application possibilities of the present invention ensue from the description below of an embodiment referring to the drawings. In this context, all the described and/or depicted features, either on their own or in any meaningful combination, constitute the subject matter of the present invention, also irrespective of their compilation in the claims or the claims to which they refer.

For clarity, it is to be understood that the word “proximal” when concerning orientation in the human body refers to a direction relatively closer to the center of the body than another part. The opposite of “proximal” is “distal”. For example, the proximal part of a femur bone is closer to a hip joint, and a distal end of the femur bone is closer to a knee joint. When referring to structure of the surgical assistance device, the word “proximal” refers to a position closer to the surgeon or clinician wearing the device and “distal”, which is opposite of “proximal”, refers to a position spaced farther from the surgeon or clinician wearing the device.

Certain terminology is used in the following description for convenience only and is not limiting. Unless specifically set forth herein, the terms “a,” “an” and “the” are not limited to one element, but instead should be read as meaning “at least one.” The terminology includes the words noted above, derivatives thereof and words of similar import.

It also should be understood that the terms “about,” “approximately,” “generally,” “substantially” and like terms, used herein when referring to a dimension or characteristic of a component of the invention, indicate that the described dimension/characteristic is not a strict boundary or parameter and does not exclude minor variations therefrom that are functionally similar. At a minimum, such references that include a numerical parameter would include variations that, using mathematical and industrial principles accepted in the art (e.g., rounding, measurement or other systematic errors, manufacturing tolerances, etc.), would not vary the least significant digit.

The present invention will be described in detail by way of examples with reference to the attached drawings. Throughout this description, the preferred embodiments and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s). The following description is provided to enable any person skilled in the art to make and use the invention and sets forth the best modes contemplated by the inventor of carrying out his invention. Various modifications, however, will remain readily apparent to those skilled in the art without departing from the spirit and scope of the invention, which is defined by the accompanying claims.

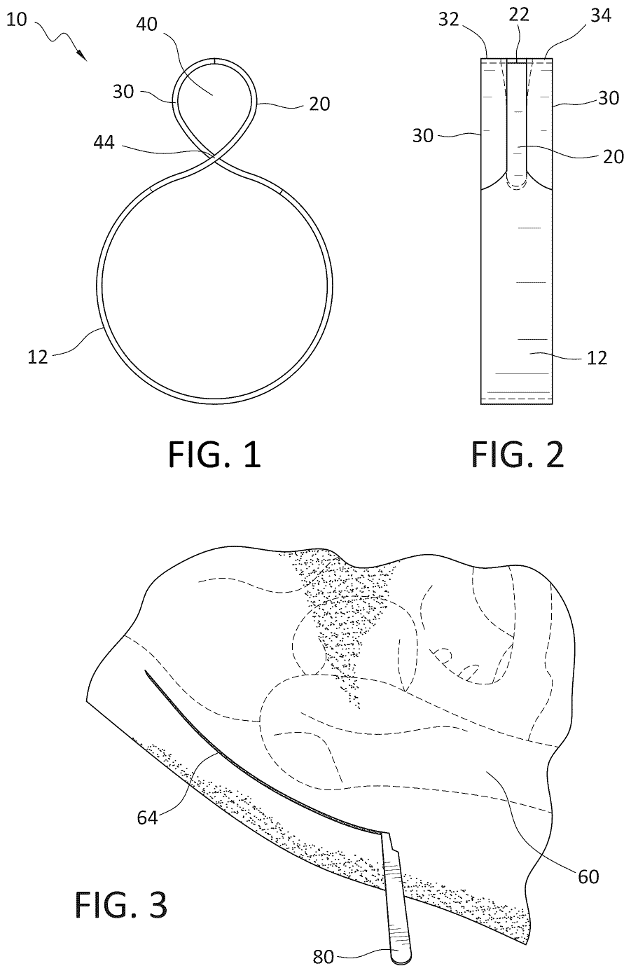

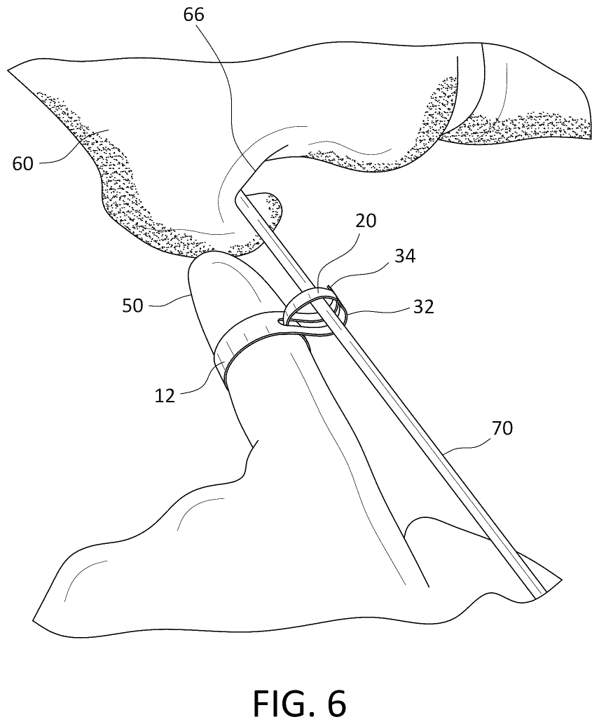

Referring now to the drawings, wherein the showings are for purposes of illustrating embodiments of the subject matter herein only and not for limiting the same, FIG. 1 illustrates an embodiment of a surgical assistance device 10. The surgical assistance device 10 is intended in one advantageous deployment to facilitate placement and orientation of a guidewire 70 (FIG. 5) into a patient's bone 60 (e.g., a femur bone, as shown in FIG. 6) in an orthopedic surgery.

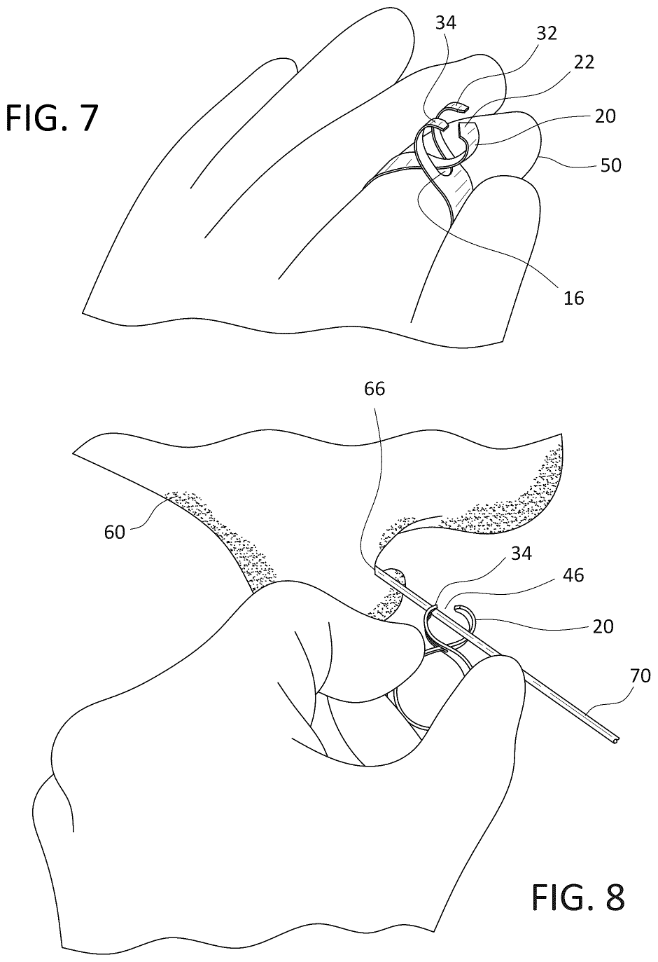

As shown in FIG. 1, the surgical assistance device 10 may include a band 12 (e.g., a finger band) sized to be worn on a finger 50 (as shown in FIG. 7), for example, a surgeon's or other practitioner's or clinician's index finger. In some embodiments, as shown in FIG. 1, the band 12 may be a substantially ring-shaped band 12. Additionally, or alternatively, the shape of the band 12 (or portions thereof) may be oval, polygonal or another shape structure configured to be worn on the finger 50.

In some embodiments, the band 12 may be sized to fit on one finger 50 (i.e., the band 12 may fixed and not adjustable). Additionally, or alternatively, the band 12 may be adjustable to fit on any finger 50. In some embodiments, the adjustability of the band 12 may result from the materials forming the band 12. For example, the band 12 may be formed from a flexible material that allows for adjusting the band 12 with minimal to no effort. As another alternative, a portion of the band 12 may be expandable to adjust the ring size to fit on any finger 50. Additionally, or alternatively, the band 12 may be formed from a stiff, yet deformable, material, which may be adjusted to fit on any finger 50. In some embodiments, the ring-shaped band 12 may be discontinuous and have one or more gaps. In any embodiment, the surgical assistance device 10 may be made of a material that may withstand multiple surgical instrument cleaning cycles, for example, at temperatures of 270° F. or above. For example, the material forming the surgical assistance device 10 may be a metal, such as but not limited to, stainless steel or aluminum or alloys thereof.

With continued reference to FIG. 1, the surgical assistance device 10 includes at least one channel 40 at an upper end of the band 12. The channel 40 is shaped and sized for a guidewire 70 (FIG. 5) to be threaded (or inserted) therethrough and into a patient, for example, during a medical procedure.

In some embodiments, the band 12 and the channel 40 may be separate pieces operably connected to form the surgical assistance device 10 (e.g., a unibody surgical assistance device 10). The materials forming the channel 40 may differ from the materials forming other parts of the surgical assistance device 10 (e.g., the band 12). The materials forming two or more parts of the surgical assistance device 10 may also be the same, as described in the exemplary embodiments herein.

Additionally, or alternatively, the surgical assistance device 10 may include one or more gaps. In one embodiment, a surface of the band 12 and/or the channel 40 may be gapped (i.e., included a gap). A first gap in the band 12 may be formed at a lower end of the band 12 opposite a surface where an upper part of the band 12 and lower part of the channel 40 are contiguous. A second gap in the channel 40 may be formed at an upper part of the channel 40 opposite the surface where the lower part of the channel 40 and upper part of the band 12 are contiguous. In some embodiments, the first gap and the second gap may be aligned at opposite ends of the surgical assistance device 10. The first and second gap may have the same or different sizes. The first gap should be sized to retain the finger therebetween at least during operation (i.e., when the surgical assistance device 10 is used). The second gap should be sized to retain the guidewire 70 within the channel 40 during operation. In some embodiments, opposed ends at the upper part of the channel 40, where the second gap is formed, may be movable towards each other to reduce the channel 40 opening for receiving the guidewire 70 or away from each other to increase such opening. It should be appreciated that moving the opposed ends towards each other to touch or otherwise connect the opposed ends reduces or eliminates the gap (e.g., the first and/or second gap). An adjustable gap (e.g., the first gap and/or second gap) provides many benefits. For example, a single band 12 having a gap would accommodate fingers of different sizes. Adjusting the second gap (as described above) would allow for a single embodiment of the channel 40 to accommodate guidewires 70 of different sizes (gauges).

With continued reference to the figures, and with reference now to FIG. 2, the channel 40 may be formed from the same material forming the band 12, such as a metal, or more preferably stainless steel or aluminum or an alloy thereof. As shown in FIG. 2, a first clasp jaw 20 and a second clasp jaw 30 extend substantially radially outwardly from the band 12 at a fulcrum 44. The first clasp jaw 20 has its proximal end at the band 12 and has a curved prong end 22 with a distal tip. The second clasp jaw 30 comprises a first prong end 32 and a second prong end 34 spaced apart from the first prong end 32. Each of the first prong end 32 and the second prong end 34 have proximal ends at the band 12 and terminate at distal tips. The prong end 22 of the first clasp jaw 20 fits between the first prong end 32 and second prong end 34 of the second clasp jaw 30 in the space between the first prong end 32 and the second prong end 34. In an initial closed configuration, the prong end 22 of the first clasp jaw 20 is fitted between the first prong end 32 and second prong end 34 of the second clasp jaw 30. In this closed configuration, the channel 40 is formed between the first clasp jaw 20 and the second clasp jaw 30, which channel 40 is sized to threadably receive the guidewire 70 to be inserted into the patient.

It should be appreciated that in the embodiment shown in FIG. 2, the surgical assistance device 10 is integrally formed from a strip of a same material that is cut to form the first clasp jaw 20 at one end and to form a slot 16 and second clasp jaw 30 at the opposite end. The strip is then bent to form the curved finger band 12, the prong end 22 and first and second prong ends 32, 34, and a base portion (proximal end portion) of the prong end 22 of the first clasp jaw 20 is seated and held in the slot 16 at the fulcrum 44. Other clasp mechanisms are envisioned, including but not limited to, lobster claw clasps or spring ring clasps known in the jewelry industry. Particularly if other clasp mechanisms are employed, the band 12 could be formed of a different material from the clasp mechanism.

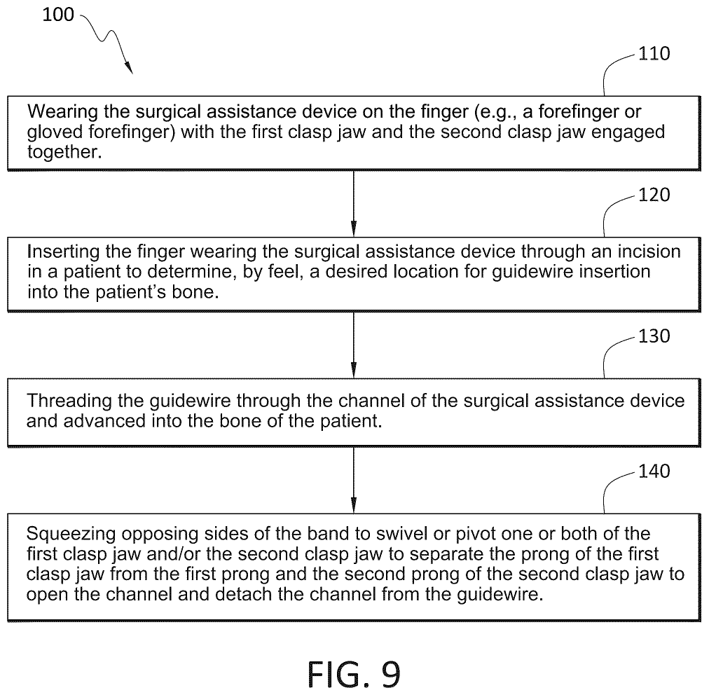

With continued reference to the figures, and with reference now to FIG. 3 through FIG. 8, a method of using the surgical assistance device 10 is illustrated schematically. A flowchart of a method of using the surgical assistance device 10 is provided in FIG. 9. FIG. 3 through FIG. 8 schematically illustrates installing a guidewire into the bone 60, for example, at the trochanteric entry point often used for fixation of femur factures.

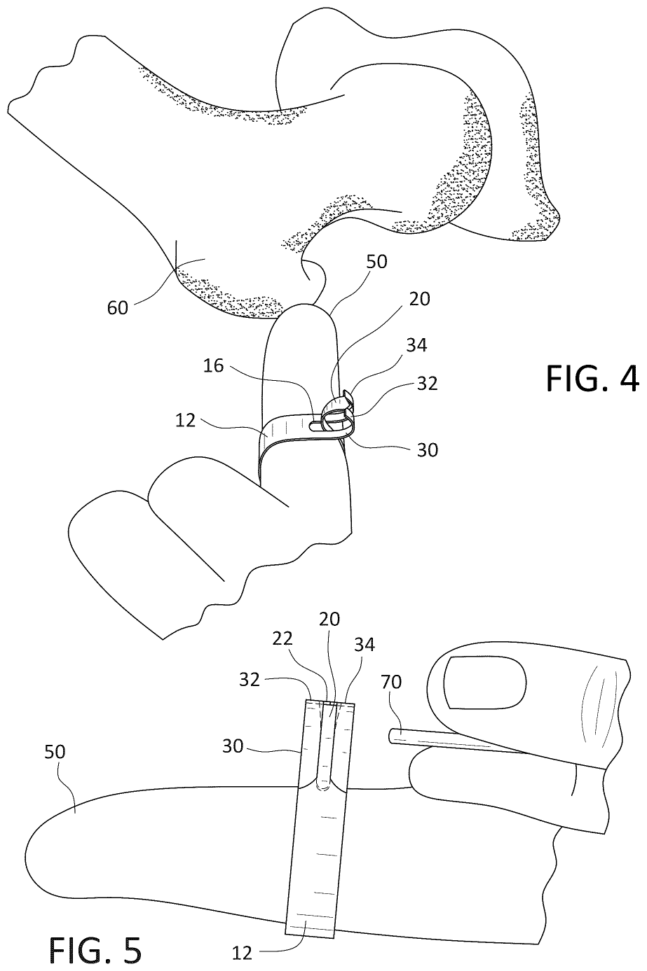

As shown in the exemplary embodiment of FIG. 3, a surgeon or other clinician cuts a suitable incision 64 with a scalpel 80 to provide access to a patient's bone 60, such as a femur, into which a guidewire 70 will be installed as part of a fixation of a femur fracture. As shown in the exemplary embodiment of FIG. 4, the surgeon or other clinician wears the band 12 of the surgical assistance device 10 on his or her finger 50, preferably the forefinger. The surgical assistance device 10 is so worn as the surgeon or other clinician palpates the patient's bone through the surgical incision 64 to locate the desired entry point for installing the guidewire 70. The exemplary embodiment of FIG. 5 shows the guidewire 70 threaded into the channel 40 between the first clasp jaw 20 and second clasp jaw 30.

The exemplary embodiment of FIG. 6 illustrates the drilling of the guidewire 70 into the patient's bone 60 while the guidewire 70 is threaded through the surgical assistance device 10. The surgeon or clinician may retain his or her finger 50 in contact with the patient's bone 60 while threading and drilling the guidewire 70 into the patient's bone 60 at the desired entry point. The surgical assistance device 10 acts as a mechanical conduit for the guidewire 70, directing the guidewire 70 at the desired entry point and in the desired orientation and direction during the surgical procedure. All the while, the surgeon or clinician may maintain his or her finger 50 in contact with the patient's bone 60. The surgical assistance device 10 maintains the guidewire 70 in a direction that is generally or substantially parallel to the finger 50 on which the band 12 is worn.

The prong end 22 of the first clasp jaw 20 and the first and second prong ends 32, 34 of the second clasp jaw 30 are movable at the fulcrum 44 between them. FIG. 7 and FIG. 8 show that the distal end of the prong end 22 of the first clasp jaw 20 may be spaced apart from the distal ends of the first prong end 32 and second prong ends 34 of the second clasp jaw 30 in response to squeezing force applied to the opposite outer circumferential surfaces of the band 12. The squeezing force may be so applied while the band 12 remains on the finger 50, for example, as shown in FIG. 7.

Additionally, or alternatively, the squeezing force may be so applied after the band 12 has been removed from the finger 50, as shown in FIG. 8. Once the prong ends 22 and 32, 34 are sufficiently separated, the channel 40 formed by the first clasp jaw 20 and second clasp jaw 30 may be opened sufficiently to create a space 46 to release the surgical assistance device 10 away from the guidewire 70 and extract the surgical assistance device 10 out of and/or away from the patient.

With continued reference to the figures, and with reference now to FIG. 9, an exemplary embodiment of a method 100 of performing a medical (e.g., a surgical) procedure using exemplary embodiments of the surgical assistance device 10 to place and advance the guidewire 70 is provided.

It should be noted that steps, recited below or in any claim, do not necessarily need to be performed in the order in which they are recited. Those of ordinary skill in the art will recognize variations in performing the steps from the order in which they are recited. In addition, the lack of mention or discussion of a feature, step or component provides the basis for claims where the absent feature or component is excluded by way of a proviso or similar claim language.

In step 110, the surgical assistance device 10 may be worn on the finger 50 (e.g., a forefinger or gloved forefinger) with the first clasp jaw 20 and the second clasp jaw 30 engaged together.

In step 120, the finger 50 wearing the surgical assistance device 10 may be inserted through the incision 64 to determine by feel a desired location for guidewire 70 insertion into a bone 60 of the patient.

In step 130, the guidewire 70 may be threaded through the channel of the surgical assistance device 10 and advanced into the bone 60 of the patient.

The method 100 may further include in step 140, squeezing opposing sides of the band 12 to swivel or pivot one or both of the first clasp jaw 20 and/or the second clasp jaw 30 to separate the prong of the first clasp jaw 20 from the first prong and the second prong of the second clasp jaw 30 to open the channel 40, which may detach the channel 40 from the guidewire 70.

In some embodiments, the prong end 22 of the first clasp jaw 20 opposingly faces the first prong end 32 and the second prong end 34 of the second clasp jaw 30 when said prong end 22 and said first prong end 32 and second prong end 34 may be separated from one another. Additionally, or alternatively, the proximal ends of the first clasp jaw 20 and the second clasp jaw 30 may extend substantially radially outwardly from the band 12. In this configuration, the band 12 defines a center axis, wherein the channel 40 defines a channel center axis, and the channel center axis is substantially parallel to the center axis of the band 12. The guidewire 70 then may be threaded in the direction of the channel center axis, which is a direction substantially parallel to the finger 50 of the surgeon or clinician wearing the band 12 of the surgical assistance device 10.

### REFERENCE NUMERALS

| | |
|---|---|
| 10 | surgical assistance device |
| 12 | band |
| 16 | slot opening |
| 20 | first clasp jaw |
| 22 | prong end |
| 30 | second clasp jaw |
| 32 | first prong end |
| 34 | second prong end |
| 40 | channel |
| 44 | fulcrum |
| 46 | space |
| 50 | finger |
| 60 | femur bone |
| 64 | surgical incision |
| 66 | desired entry point for guidewire |
| 70 | guidewire |
| 80 | scalpel |
| 100 | method of performing medical prodcedure with surgical |
| | assistance device |
| 110 | method step |
| 120 | method step |
| 130 | method step |
| 140 | method step |

What is claimed is:

1. A surgical assistance device, comprising:
a substantially ring-shaped band;
a first clasp jaw extending outwardly from the band and having a prong end;
a second clasp jaw extending outwardly from the band and having a first prong end and a second prong end, said first prong end and said second prong end being spaced apart, wherein at least a portion of the first clasp jaw is configured to be received between the first prong end and the second prong end of the second clasp jaw, with the first clasp jaw and the second clasp jaw when so engaged together forming a channel configured to receive a guidewire; and
wherein one or more of the prong end of the first clasp jaw and the first prong end and the second prong end of the second clasp jaw are movable in relation to one another.

2. The surgical assistance device of claim 1, wherein the band defines an elongated slot.

3. The surgical assistance device of claim 2, wherein a proximal portion of the first clasp jaw is swivelably held in the elongated slot of the band.

4. The surgical assistance device of claim 3, wherein the first prong end and the second prong end of the second clasp jaw have proximal ends extending from the band at opposite sides of the elongated slot.

5. The surgical assistance device of claim 2, wherein the proximal portion of the first clasp jaw is pivotably held in the elongated slot of the band.

6. The surgical assistance device of claim 1, wherein the prong end of the first clasp jaw and the first prong end and the second prong end of the second clasp jaw are separable from one another to open the channel.

7. The surgical assistance device of claim 6, wherein the first clasp jaw and/or the second clasp jaw swivel or pivot when opposing external circumferential surfaces of the band are squeezed.

8. The surgical assistance device of claim 6, wherein the prong end of the first clasp jaw opposingly faces the first prong end and the second prong end of the second clasp jaw when said prong end and said first prong end and second prong end are separated from one another.

9. The surgical assistance device of claim 1, wherein proximal ends of the first clasp jaw and the second clasp jaw extend substantially radially outwardly from the band.

10. The surgical assistance device of claim 1, wherein the band defines a center axis, wherein the channel defines a channel center axis, and the channel center axis is substantially parallel to the center axis of the band.

11. The surgical assistance device of claim 1, wherein the band, the first clasp jaw and the second clasp jaw are integrally formed of a same material.

12. The surgical assistance device of claim 1, wherein the band, the first clasp jaw and the second clasp jaw are formed of a metal that can withstand medical sterilization at a temperature of about 270° F. or above.

13. The surgical assistance device of claim 10, wherein the band, the first clasp jaw and the second clasp jaw are formed of a material selected from the group consisting of: stainless steel, aluminum and alloys thereof, and medical-grade plastic.

14. The surgical assistance device of claim 1, wherein the substantially ring-shaped band is discontinuous.

15. A method of placing and advancing a surgical guidewire, comprising:
wearing a surgical assistance device according to claim 1 on a forefinger or gloved forefinger, with the first clasp jaw and the second clasp jaw engaged together;
inserting the forefinger or gloved forefinger through an incision in a patient to determine by feel a desired location for guidewire insertion into a bone of the patient;
threading the guidewire through the channel of the surgical assistance device and advancing the guidewire into the bone of the patient;
squeezing opposing sides of the band to swivel or pivot one or both of the first clasp jaw and the second clasp jaw to separate the prong of the first clasp jaw from the first prong and the second prong of the second clasp jaw to open the channel; and
detaching the channel from the guidewire.

16. The method of claim 15, wherein the prong end of the first clasp jaw opposingly faces the first prong end and the second prong end of the second clasp jaw when said prong end and said first prong end and second prong end are separated from one another.

17. The method of claim 15, wherein proximal ends of the first clasp jaw and the second clasp jaw extend substantially radially outwardly from the band.

18. The method of claim 15, wherein the band defines a center axis, wherein the channel defines a channel center axis, and the channel center axis is substantially parallel to the center axis of the band.

19. The method of claim 18, wherein the guidewire is threaded in the direction of the channel center axis.

20. The method of claim 19, wherein the guidewire is threaded in a direction substantially parallel to the forefinger.

21. A surgical assistance device, comprising:
a substantially ring-shaped band;
a first clasp jaw extending outwardly from the band and having a prong end;
a second clasp jaw extending outwardly from the band and having a first prong end, wherein the prong end of the first clasp jaw opposingly faces the first prong end of the second clasp jaw, with the first clasp jaw and the second clasp jaw when engaged together or in contact with one another forming a channel configured to receive a guidewire; and
wherein one or more of the prong end of the first clasp jaw and the first prong end of the second clasp jaw are movable in relation to one another.

22. The surgical assistance device of claim 21, wherein the band defines an elongated slot.

23. The surgical assistance device of claim 22, wherein a proximal portion of the first clasp jaw is swivelably or pivotably held in the elongated slot of the band.

24. The surgical assistance device of claim 21, wherein the band defines a center axis, wherein the channel defines a channel center axis, and the channel center axis is substantially parallel to the center axis of the band.

25. The surgical assistance device of claim 21, wherein the band, the first clasp jaw and the second clasp jaw are integrally formed of a same material.

26. The surgical assistance device of claim 21, wherein the band, the first clasp jaw and the second clasp jaw are formed of a metal that can withstand medical sterilization at a temperature of about 270° F. or above.

27. The surgical assistance device of claim 21, wherein the band is discontinuous.
